(19) United States
(12) Patent Application Publication
(10) Pub. No.: US 2003/0036052 A1
(43) Pub. Date: Feb. 20, 2003

(54) Sensor for analyzing components of fluids

(75) Inventors: Michael J. Delwiche, Winters, CA (US); Daniel M. Jenkins, Davis, CA (US); Edward J. DePeters, Davis, CA (US); Robert H. BonDurant, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA

(21) Appl. No.: 10/209,455

(22) Filed: Jul. 30, 2002

Related U.S. Application Data
(63) Continuation-in-part of application No. 09/839,939, filed on Apr. 19, 2001.
Application No. 09/839,939 is a division of application No. 09/349,814, filed on Jul. 9, 1999, now Pat. No. 6,287,851.

(51) Int. Cl. C12M 1/34

(52) U.S. Cl. 435/4; 435/287.5; 436/148; 422/98

(57) ABSTRACT

The present invention provides methods and sensors for assaying components of fluid samples by measuring pressure changes. In particular, the invention provides methods to assay for components in fluids with or without the aid of enzymatic reactions. The invention also provides a modified sensor that has an immersible pressure monitor/gas-containing portion unit and is capable of detecting pressure changes with higher sensitivity.

### CROSS-REFERENCES TO RELATED APPLICATIONS

This application is a continuation-in-part of U.S. application Ser. No. 09/839,939, which is a divisional application of application Ser. No. 09/349,814, issued as U.S. Pat. No. 6,287,851, the disclosures of which are incorporated herein by reference in their entirety for all purposes.

### FIELD OF THE INVENTION

This invention generally relates to methods and devices for assaying components of fluid samples. More specifically, the invention relates to methods for determining concentrations of components in fluid samples based on vapor pressure changes, and improved devices for this purpose.

### BACKGROUND OF THE INVENTION

Gasometric techniques were among the first technologies available to analytical chemists. Knop (Fresenius Zeitschriftfuir Analytische Chemie. 9:225-231,1870) estimated the amount of ammonia and urea in solution by measuring the amount of N₂ and CO₂ gas evolved from reactions with hypobromite. In one of the first recorded analytical uses of enzymes, Partos (Biochemische Zeitschrift. 103:292-298,1920) was able to measure urea in solution with a u-tube manometer after hydrolyzing the urea to ammonia and CO₂ with urease (Enzyme Commission, or EC, #3.5.1.5). Manometric instruments were common in chemistry laboratories through the middle of the 20th century, and were used for a variety of clinical applications as well as in early experiments elucidating the mechanisms of cellular respiration (Dixon, M., Manometric Methods. Cambridge University Press, London, UK, 1934). These instruments were gradually phased out after the development of sensors suitable for optical and electrochemical analysis.

For some applications, however, manometric methods have advantages over newer optical and electrochemical methods. Some fluids, for example milk, are chemically complex media containing a variety of components that may cause optical or electrochemical interference with a sensor, and have a high solids content that may cause the failure of delicate sensor components. The development of inexpensive microfabricated pressure monitors has led to the automation of manometric techniques which are effective to difficult working fluids because the sensing element does not need to come into direct contact with the sample.

Using the same concepts as Partos, supra, a manometric sensor for measurement of urea has been developed for analyzing biological fluids such as blood, milk, or urine (see U.S. Pat. No. 6,287,851). The sensor includes a chamber having a liquid containing portion and a vapor containing portion, with the two portions in fluid communication through a porous membrane. The fluid sample occupies the liquid containing portion and urea in the fluid is subsequently hydrolyzed by an enzyme. A pressure monitor, another component of the sensor, measures the pressure changes in the vapor containing portion due to the release of CO₂ from the fluid during urea hydrolysis. The concentration of urea in the fluid sample can then be determined based on the amount of CO₂ released. Because it is fast, accurate, and inexpensive, this technique is particularly useful for the dairy industry to monitor the level of urea in milk, which is well correlated to the level of urea in blood and urine, in order to balance the feed rations for optimal nitrogen efficiency (see, Jenkins and Delwiche, Biosensors & Bioelectronics. 17(6-7):557-563, 2002).

Other components of fluid samples can also be measured by a sensor of this type employing the same principle. For instance, most amino acids and some β-ketoacids such as acetoacetate may be enzymatically decarboxylated and measured through the volatilization of CO₂. Similarly, reactions depleting dissolved gases such as oxygen may be measured by the change in vacuum in a closed cell. Glucose and lactose are examples of the many important metabolic carbohydrates which may be measured through enzymatic oxidation.

There were, however, several limitations associated with these closed-reactor type of manometric sensors. A principal disadvantage was the fact that they could only be used for the analysis of discrete samples in a sealed system. Another limitation of these manometric sensors arose from practical considerations of dimensioning the volume of the vapor containing portion relative to the volume of the liquid containing portion. For effective mass transfer and reproducible sensitivity, the gaseous volume was required to be relatively large (greater than ⅕of the liquid volume). This relatively large gaseous volume caused a loss of sensitivity from the theoretical maximum.

The present invention seeks to solve these problems by enclosing the vapor containing portion and the pressure monitor with a porous membrane. Soluble gases could then move across the membrane to and from the fluid sample, but the gas phase would be held inside the cavity due to the surface tension of the fluid on the membrane. The pressure within the cavity, i.e., the pressure within the vapor containing portion, could then be independent of the pressure in the fluid sample, and the entire pressure monitor could be immersed in the fluid. Furthermore, the volume of the vapor containing portion could be made constant and much smaller so that the sensitivity of the sensor could be reproducible and as high as possible.

### BRIEF SUMMARY OF THE INVENTION

One principal disadvantage of previously described manometric sensors as compared to other technologies was the fact that they could only be used for the analysis of discrete samples in a sealed system. Another limitation of manometric sensors arose from practical considerations of dimensioning the headspace gas volume relative to the sample volume. For effective mass transfer and reproducible sensitivity, this volume was required to be relatively large (>0.2 times the sample volume). As will subsequently be discussed, this caused a loss of sensitivity from the theoretical maximum.

The present invention provides devices and methods for measuring the presence and changes in the concentration of selected components within chemically complex media. The invention is useful for determining a single component from liquids that contain a variety of components that may cause optical or electrochemical interference with a sensor. The present invention is also of use in determining a component of a fluid that has a high solids content that may cause the failure of delicate sensor components. Moreover, the invention provides devices and methods for the automation of manometric techniques which are effective under a wide range of conditions because the sensing element does not come into direct contact with the sample.

In a first aspect, the present invention provides a sensor for assaying a component in a fluid. The sensor includes a housing that defines a chamber. The chamber has an upper and lower section. The upper section includes an opening that communicates with the atmosphere and that opening is covered with a permeable membrane, which has an inside surface. The chamber also includes a lower section in which a piezoresistive pressure transducer is located. The transducer is covered with a transducer coating material having an upper surface. The inside surface of the membrane and the upper surface of the coating material define a gaseous cavity. The sensor housing is optionally covered with an encapsulant that prevents liquid and gas leakage into the sensor.

In some embodiments of the present invention, the sensor is in a vessel that is sealed against the ambient environment. The vessel includes the fluid to be assayed and optionally further comprises an enzyme source adapted to provide an enzyme for which the component or an intermediate product of the component is a substrate. In some embodiments, the vessel has an inlet adapted to admit one or more substance into the fluid. In some embodiments, the vessel has no inlet and the enzyme is placed within the liquid containing portion of the chamber before the admission of the fluid. In some embodiments, the enzyme is immobilized. In some embodiments, the chamber further contains an agitator sufficient to cause equal distribution of all components in the fluid.

In another embodiment, the component measured is urea, and the enzyme is urease. The fluid sample comprises blood, milk, or urine, and the pressure changes detected within the vapor containing portion is due to the volatilization of dissolved CO₂. In another embodiment, the component measured is glucose or lactose, and the enzyme used is glucose oxidase. Exemplary fluid samples include blood, milk, or urine. The pressure changes detected within the vapor containing portion are due to the depletion of oxygen as a result of the enzymatic oxidation of the substrate. In other preferred embodiments, the sensor is used to estimate atmospheric humidity.

The sensor is used in a batch or continuous mode. In an exemplary embodiment, the sensor is used to continuously monitor the level of dissolved CO₂. As more CO₂ is delivered into the fluid, the pressure monitor immersed in the fluid records the pressure changes, thereby monitoring the changing CO₂ level in the fluid.

The second aspect of the present invention is a method for assaying a liquid for the presence of a component in the liquid. The method includes: (a) contacting the liquid with a sensor that includes a gaseous chamber in contact with a pressure transducer, and the gaseous chamber is separated from the liquid by a permeable membrane; and (b) detecting a change in pressure in the gaseous chamber with said pressure transducer. The change in pressure is due to the presence of the component in the liquid.

Other aspects, advantages and objects of the invention will be apparent from the detailed description that follows.

### BRIEF DESCRIPTION OF THE DRAWINGS

FIG. 1 is a schematic diagram of an immersible pressure monitor/vapor-containing portion unit.

FIG. 2 is a schematic diagram of fluid handling hardware for an exemplary sensor.

FIG. 3 is a schematic of an exemplary setup for the experimental determination of urea and glucose.

FIG. 4 is a graphical display of the vacuum (Pₐₜₘ-Pₛₐₘₚₗₑ) observed in a glucose sensor, at 21° C., during agitation of 5 mM lactose sample previously hydrolyzed with β-galactosidase.

FIG. 5 are the chemical pathways used for the measurement of glucose and lactose. Depletion of O₂ during the enzymatic oxidation of β-D-glucose to β-D-gluconolactone is measured as vacuum.

FIG. 6 is a standard curve of sensor response with glucose at 25° C. (Vacuum=0.597; Glucose −0.319; R2=0.9849; S.E.=0.23 mM).

FIG. 7 is a graphical display of the observed vacuum change in assay of 5 mM lactose standard against incubation time with β-galactosidase, at 23° C.

FIG. 8 is a standard curve of sensor response for lactose at 25° C. (Vacuum=0.572; Lactose −0.169; R2=0.9892; S.E.=0.19 mM).

FIG. 9 is a comparison of lactose determination in milk by manometric sensor with values determined by FTIR spectroscopy (R2=0.45; root mean squared deviation of sensor prediction from FTIR instrument is 8.1 mM, or about 5.8% of the average observation).

FIG. 10 is a graphical display of the observed (·) and predicted(−) sensitivity of a glucose sensor at 25° C. for different ratios of gas to sample volume in a fixed volume reactor.

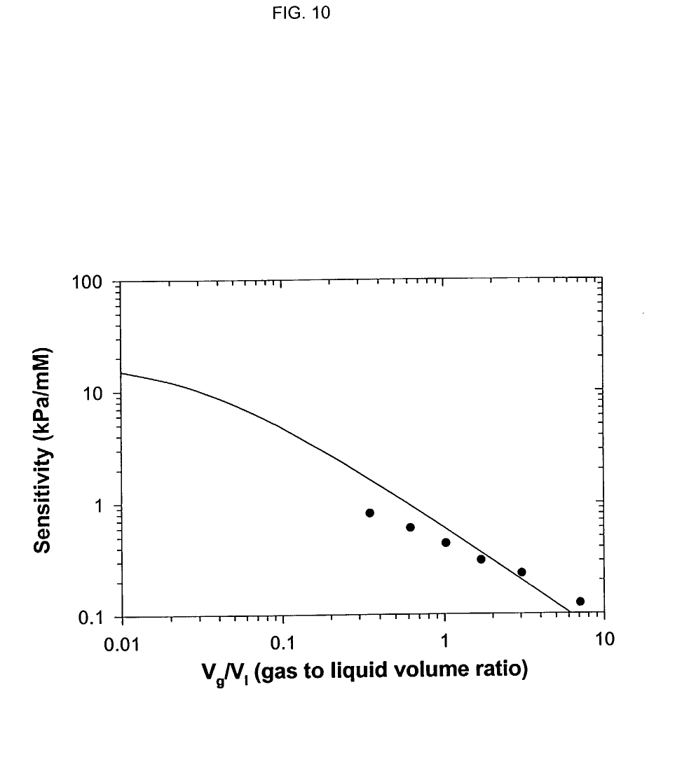

FIG. 11 is a display of a typical transient response to immersion of pressure monitor in distilled water. Monitor is immersed at time 0, and withdrawn from the water after 3 minutes (Dry bulb temperature=21° C., wet bulb temperature=14° C., atmospheric pressure =101.4 kPa).

FIG. 12 is a graphical display of the observed pressure change after immersion in distilled water against predicted value based on psychrometric observations (Y=0.9544X+0.086; R2=0.9993; root mean squared difference=0.038 kPa).

FIG. 13 is a comparison of relative humidity estimated from manometric sensor to that estimated by psychrometric observations (Y=0.895X+3.46; R2=0.9737; root mean squared difference=1.1%).

FIG. 14 Continuous monitoring of cavity pressure in immersible pressure monitor during multiple injections of bicarbonate into sample at 28° C. Each vertical dashed line represents a step change of approximately 1 mM in dissolved CO₂.

FIG. 15 is a standard curve of pressure observed in immersible pressure monitor against urea concentration at 28° C. (Pressure=1.022 [Urea] +0.015; R2=0.9917; standard error=0.086 mM).

FIG. 16 Are typical profiles of pressure over time observed during the assay of urea standards, indicating that the 15-minute pressure development time was not sufficient to reach equilibrium.

FIG. 17 is a standard curve of vacuum observed with an immersible pressure monitor against glucose concentration at 28° C. (Vacuum=0.1012 [Glucose] +0.02; R2=0.9644; standard error=0.76 μM).

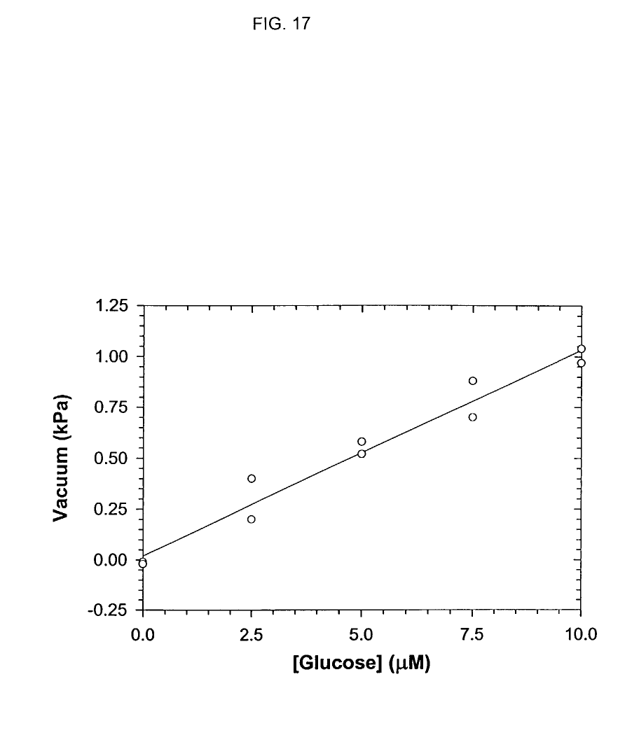

FIG. 18 are typical profiles of vacuum over time observed during the assay of glucose standards, suggesting that the 60-minute pressure development time may not have been sufficient to reach equilibrium.

FIG. 19 are observed and predicted values of sensitivity for manometric sensors measuring urea and glucose.

### DETAILED DESCRIPTION OF THE INVENTION

Introduction

The present invention relates to methods and sensors for assaying components of fluid samples. In particular, this invention provides an immersible sensor and methods of using that sensor for assaying a component in a fluid. The sensor of the present invention is made immersible by configuring the sensor such that the pressure monitor and the vapor-containing gaseous cavity are separated from the fluid by a membrane that is permeable to a gas. The invention also enhances the versatility of prior detection technology by expanding the use of the sensor to measuring organic substrates (e.g., saccharides, urea, etc.) in a fluid sample by converting them to a gas, or by degrading them in a manner that requires a gas as a consumed reactant; both processes are detectable by the sensor of the invention. Using the methods and devices of the invention, the presence of organic substrates in a fluid can be detected, and their amount can be reliably quantified. Furthermore, the methods and device of the invention are operable under a broad range of conditions, including those appropriate for the enzymatic and/or chemical conversion of the organic substrates into a detectable species. Moreover, in addition to providing methods and devices to detect organic substrates in a liquid, the present invention provides methods to determine water vapor pressure or the amount of CO₂ dissolved in a fluid sample.

The Device

In a first aspect, the present invention provides a pressure sensor that is immersible in a fluid. The sensor measures changes in gas pressure from water vapor or soluble gases in the fluid. Alternatively, the sensor measures a decrease in gas pressure in the fluid.

The sensor includes a housing having a chamber therein. The chamber has an upper and lower section. The upper section includes an opening that communicates with the atmosphere and that opening is covered with a permeable membrane, which has an inside surface. The chamber also includes a lower section in which a piezoresistive pressure transducer is located. The transducer is covered with a transducer coating material having an upper surface. The inside surface of the membrane and the upper surface of the coating material define a gaseous cavity. The sensor housing is optionally covered with an encapsulant that prevents liquid and gas leakage into the sensor.

The sensor is typically connected to instrumentation for recording and/or displaying the information transmitted by the pressure tranducer. Those of skill in the art will appreciate that the sensor can be used with an array of wiring and instrumentation configurations.

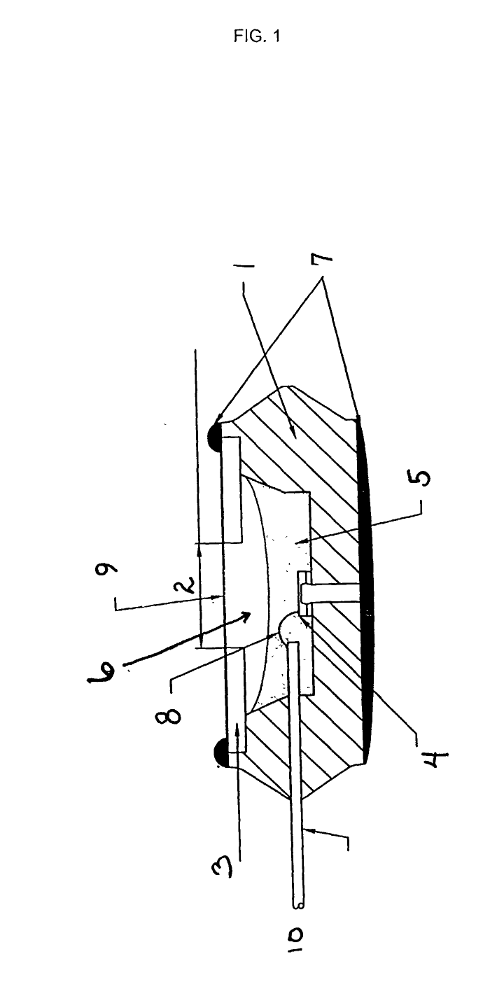

An exemplary sensor of the invention is understood by reference to FIG. 1. The sensor detects the presence of a selected component in the fluid by a change in pressure across pressure transducer 4.

The transducer is located within housing 1. The housing may be made of any appropriate material, which is preferably inert under the conditions used to assay the fluid with the sensor. Thus, the sensor housing may be made from metals, ceramics, plastics, glasses and the like.

The housing defines a chamber that is open to the atmosphere at one face. The opening to the chamber is covered by a permeable membrane 9, forming gaseous cavity 6 between the surface of the membrane facing the inside of the cavity and a coating layer 5 formed over pressure transducer 4. The remainder of the cavity is sealed by plates 3. The chamber may be of any convenient size, however, the inventors have recognized that the dynamic properties of the device are improved by the use of smaller cavity volumes. Because the volume of the gaseous cavity can be made constant and much smaller than previous sensors, the sensor is highly sensitive and provides reproducible results.

The device of the invention is optionally coated with encapsulant 7 to seal any openings between the gaseous cavity and the ambient atmosphere that may have developed during assembly. Exemplary encapsulants include plastics and resins such as a two part epoxy coating.

The device is typically attached to instrumentation through cable 10, which is connected by wire connection 8 to transducer 4. The sensor is connected to the instrumentation through a wiring-means, such as a four-conductor shielded cable. In operation, the sensor is powered by a power source such as a 12 VDC source. The signal from the sensor is preferably amplified by, for example, an adjustable gain instrumentation amplifier. The signal is also preferably filtered with one or more filter. Thus, in an exemplary embodiment, the signal from the transducer is filtered through a 6th order switched-capacitor low-pass Butterworth filter, and the output from this filter is filtered through a 2nd order Sallen-Key low-pass filter. The signal from the transducer, or from the filter, is preferably digitized using an analog to digital converter. The attachment of the device to various instruments using acceptable modes of attachment is well within the abilities of those of skill in the art.

The porous membrane is not limited to a pore size or range of pore sizes. The choice of an appropriate pore size for a given application will be apparent to those of skill in the art. In certain preferred embodiments, the membrane has a pore diameter of from about 0.005 micrometer to about 25 micrometers. In other preferred embodiments, the membrane has a diameter of from about 0.01 micrometer to about 1 micrometer. Exemplary membrane materials include, but are not limited to, inorganic crystals, inorganic glasses, inorganic oxides, metals, organic polymers and combinations thereof. Controlled pore membranes are presently preferred.

Inorganic oxide membranes are resistant to aggressive chemicals like acids, alkalines and solvents. These membranes are also resistant to abrasive suspensions and temperature fluctuations. Methods of making inorganic oxide membranes are known to those of skill in the art. Additionally, appropriate membranes are available commercially from sources such as Schumacher Umwelt- und Trenntechnik GmbH (Crailsheim, Germany). The membranes are available in pore sizes between 0.005-1.2 micrometers and in at least eight different geometries.

Appropriate metal membranes are available from a variety of sources such as Alternburger electronic GmbH (Seelbach, Germany). The metal membranes can be selected for desirable physical properties such as density, magnetic characteristics, conducting or insulating characteristics, heat capacity and the like.

Organic polymers that form useful membranes include, for example, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysaccharides (e.g., dextran derivatives), polysilanes, fluorinated polymers, epoxies, polyethers and phenolic resins.

Many commercially available polymer- or resin-based membranes can also be used in practicing the present invention. Moreover, commercially available membranes having well-defined pore sizes are available over a wide pore size range and composed of a number of different materials.

Presently preferred polymer- or resin-based membranes include those constructed of polymers selected from the group of polypropylene, nylon, fluorocarbon, polyester, polyethylene, polysulfone, polyether sulfone, cellulose, cellulose ester, ethyl vinyl acetate, polycarbonate, polyaramide, polyimide and combinations thereof.

The mass transfer characteristics of the device are readily altered by varying the nature of the membrane. For example, the mass transfer is increased by the use of a microfabricated membrane of a rigid material of low thickness with greater porosity and larger pores. Additional change in mass transfer characteristics is achieved by bonding a rigid membrane directly onto the silicon die with the pressure transducer, thus greatly diminishing the dimensions of the cavity for gas to diffuse through and the volume of sample required for maximum sensitivity.

The pressure transducer 4, may be of any convenient design. Transducers are devices which function generally to convert an input of one form into an output of another form or magnitude. Many types of transducers are available for converting pressure to electrical signals. In the present market, the largest number of pressure to voltage conversion devices (pressure transducers) are piezoresistive. These devices are strain sensitive rather than displacement sensitive. For pressure ranges less than a psi FS (Full Span), capacitive displacement transducers are predominantly employed. The capacitance is sensed by electrical circuits to provide an output voltage corresponding to the change in pressure. Highly developed silicon semiconductor processing techniques have given rise to the use of such material as flexible clamped transducer diaphragms which move in response to pressure and provide an output change in electrical resistance or capacitance. Such transducers are disclosed in U.S. Pat. Nos. 4,495,820; 4,424,713; 4,390,925 and 4,542,435.

In an exemplary embodiment, the device of the invention utilizes a 10 kPa commercial transducer with internal temperature compensation.

In yet another exemplary embodiment, the device of the invention is a microfabricated manometric chemical sensor with dynamic responses measured in seconds, test volumes measured in μl, and detection limits measured in ng/ml.

The device of the invention is described above by reference to exemplary embodiments of the device. Those of skill in the art will appreciate that the scope of the invention is not limited by the exemplary description, and a full range of equivalents is available for the individual components of the device as well as the manner in which they are combined to produce the device of the invention.

The Methods

In addition to the immersible manometric sensor described above, the invention also provides methods for measuring changes in gas pressure from water vapor or soluble gases. Exemplary applications of a device of the invention are set forth below. Those of skill in the art will appreciate that the methods of the invention are useful for measuring any component of a fluid that is a gas, can be converted into a gas, or which takes up a gas within a system during its conversion to another species. In exemplary embodiments, the invention provides methods to measure atmospheric humidity, monitor changes in dissolved CO₂ concentration, and determine the concentrations of solutes, such as urea and glucose in a sample.

The methods of the invention exploit the design of the device of the invention in which the gaseous volume and pressure sensor are contained within a porous membrane. Soluble gases move across the membrane, but the gas phase is held inside the cavity due to the surface tension of the sample on the membrane. The gaseous cavity pressure is independent of the pressure in the sample.

Thus, in a second aspect, the invention provides a method for assaying a liquid for the presence of a component in the liquid. The method includes: (a) contacting the liquid with a sensor that includes a gaseous chamber in contact with a pressure transducer, and the gaseous chamber is separated from the liquid by a permeable membrane; and (b) detecting a change in pressure in the gaseous chamber with said pressure transducer. The change in pressure is due to the presence of the component in the liquid.

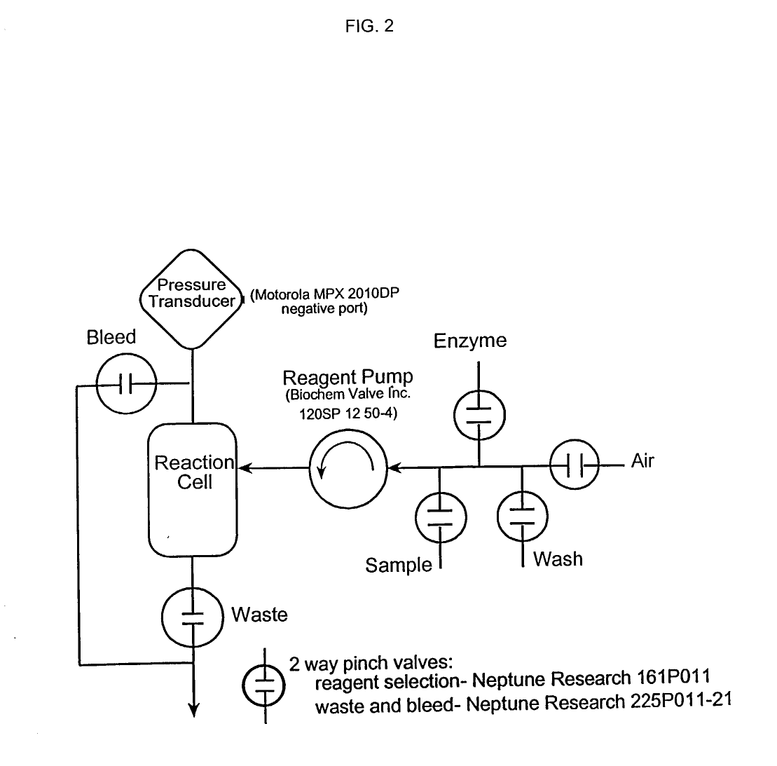

In practicing the methods of the invention, the sensor is typically contained within a vessel in which the fluid containing the gas is located. The vessel may be of substantially any convenient configuration. An exemplary configuration if provided in FIG. 2. In a further exemplary embodiment, the vessel is sealed, its contents protected from contact with the ambient environment. The vessel will typically be provided with a port through which components of the fluid, reactants, enzymes, substrates, cofactors, etc. are admitted to the sealed vessel.

The methods of the invention detect changes in vapor pressure and concentration of dissolved gases. The method may be used to detect changes in vapor pressure and concentration of any dissolved gas. The only practical limitation on the scope of gases with which the methods can be practiced is the requirement that the sensor be substantially inert to towards the gas for the duration of the measurement. Thus, the method may be used, for example to detect O₂, CO, CO₂, SO₃, H₂SO₄, SO₂, N₂, NO, NO₂, N₂O₄, H₂O and the like.

The gas may be already present in the liquid, or it may be generated prior to or during the process of the measurement. For example, it is within the scope of the invention to degrade a substrate in a manner that it generates a gas that is measured by a method of the invention. In an exemplary embodiment, the substrate is an organic molecule that is a substrate for an enzyme. Any combination of degradative enzyme and substrate may be used in the invention. Exemplary enzymes include hydrolases, decarboxylases, and dehydrogenases that cause the release of CO₂ from a substrate .

In another exemplary embodiment, the reaction of the substrate catalyzed by the enzyme takes up a gas from a fluid, creating a drop in pressure. Exemplary enzymes that catalyze reactions that take up a gas include the oxidases and oxygenases. Oxidating mechanisms provide methods that are able to detect low concentrations of substrate.

In yet another exemplary embodiment, the methods include liberating or consuming nitrogen or hydrogen gas; such methods are highly sensitive, due to the minimal solubility of the gases in the aqueous media in which most enzymatic reactions are performed.

Although the invention is exemplified by gases liberated or taken up during enzymatically catalyzed reactions of organic substrates in aqueous media, the invention is not limited to these embodiments. Thus, it is within the scope of the reaction to follow chemical reactions that are free of enzymes. Moreover, the sensor of the invention is of use to monitor reactions that occur in organic or mixed organic/aqueous systems.

The methods of the invention are appropriate for a broad range of assays. For example, the invention can be used in diagnostic applications (such as diabetes and other disorders), since metabolic carbohydrates in the citric acid cycle (using various dehydrogenases) generate carbon dioxide, which can be measured by a sensor described herein. Another application contemplated is to assess uric acid (using the enzyme uricase for animal analyses or urate oxidase for human biological fluid analyses). In the catabolism of uric acid to allantoin, which is rate enhanced by urate oxidase, carbon dioxide is again a reaction product (or byproduct). Uric acid is a contaminate in agricultural runoff, such as from the poultry industry. Uric acid analysis is also useful in assessing risk of kidney stones and gout in humans. With some variations made, practice of the invention can also be to determine the presence of an enzyme in a test sample such as soil. For example, ureas in soils lead to accelerated hydrolysis and oxidation of urea (as fertilizer) to ammonia and nitrates which leach into ground water.

Thus, the method of the invention can be broadly used to analyze a component of an enzymatically catalyzed process from a test sample. By enzymatically catalyzed process is meant that the component being analyzed is either the substrate for which the component is the enzyme or is the enzyme for which the component is the substrate. (The enzymatically catalyzed process itself, of course, can involve other moieties, such as cofactors, which will either be present in the test sample or may be supplied during practice of the method.) The test sample itself is preferably a biological fluid, but may also be in other forms when originally obtained. For example, practice of the invention for analyzing an enzyme such as urea in soil is contemplated; however, the test sample (of soil) will then be dissolved or suspended in liquid so as to facilitate the enzymatically catalyzed process.

A preferred embodiment of the present invention is a method of analyzing a component in a biological fluid. The analysis method includes the steps of providing a liquid sample of the biological fluid, contacting the sample with an enzyme for which the component is a substrate so as to form a gas as a reaction product (or take up a gas as a reactant), and detecting the amount of gas so formed.

An exemplary embodiment involves analyzing a component of milk, a representative biological fluid. An exemplary component of milk is milk urea nitrogen (MUN). In practicing this embodiment, the method includes providing a dairy milk sample and contacting the sample with urease to yield carbonate and ammonium ions. When the gas is carbon dioxide, the equilibrium is optionally shifted towards carbon dioxide by adjusting pH, and carbon dioxide vapor is detected. This detected carbon dioxide may then be related to the concentration of MUN in the dairy milk sample.

The monitoring process of the invention can be conducted continuously on a stream or to monitor a process. For example, a sensors of the invention can be used to automatically measure MUN during milking, and can thus be automated to run during an already automated milking process. The inventive sensors can complete one measurement cycle faster than the turn-around time for cows in the parlor (10 min).

The method is also of use to determine humidity (water vapor). According to the an exemplary embodiment of the invention, relative humidity can be estimated to within about 1% of predictions based upon psychrometric observations. In an exemplary embodiment, the method provides for injecting samples of air into a cavity adjacent to a wetted membrane to determine humidity in extremely small samples.

In another exemplary embodiment, the invention provides as method for measuring glucose and lactose through the consumption of oxygen during the enzymatic oxidation of those substrates. The invention provides methods for assaying many other oxidizable substrates, for example L-lactic acid through the enzyme lactate oxidase (EC #1.1.3.2).

The materials, methods and devices of the present invention are further illustrated by the examples that follow. These examples are offered to illustrate, but not to limit the claimed invention.

### EXAMPLES

Materials and Methods

a) Construction of sensor

The immersible manometric sensor (FIG. 1) was made using a 10 kPa commercial pressure transducer with internal temperature compensation (MPX2010D, Motorola Semiconductor Corp., Phoenix, Ariz., USA). To increase the area available for mass transfer, the positive port of the transducer was bored out with a #25 drill (3.8 mm diam.). A 1 μm controlled pore polycarbonate membrane (Nuclepore Cat #110410, Whatman, Kent, UK) was then fixed over the port using a commercial adhesive (Super Glue, American Glue Corp., Taylor, Mich., USA). The negative port of the sensor was covered with a two component epoxy encapsulant (Cat#832B-375ml, M.G. Chemicals, Rexdale, Ontario, Canada). To seal any cracks along the seam of the epoxy case that formed as a result of boring out the positive port in the stainless steel cover, this seam was also covered with the encapsulant.

The pressure monitor was connected to the instrumentation through a four conductor shielded cable. The bare electrical harness connecting to the monitor was coated in silicone grease (high vacuum grease, Dow Coming Corp., Midland, Mich., USA) and wrapped in a generic teflon tape to prevent wetting of the leads and gas exchange through the length of the cable. The monitor was powered with 12 VDC, and the differential signal from the monitor was amplified by an adjustable gain instrumentation amplifier made from 3 operational amplifiers (AD705, Analog Devices, Norwood, Mass., USA). The signal was then filtered with a 6^{th} order switched-capacitor low-pass Butterworth filter (MF 6CN-50, National Semiconductor, Santa Clara, Calif., USA) configured for a cutoff frequency of 21 Hz and an offset null capability. Using the spare operational amplifier from the switched-capacitor chip, the output of this filter was then filtered with a 2^{nd} order Sallen-Key low-pass filter with a cutoff frequency of 50 Hz to attenuate the 1050 Hz clock noise from the switched-capacitor circuit. The signal was then digitized through the analog to digital converter of a standard data acquisition board (DAS-16, Keithley Metrabyte, Taunton, Mass., USA). To notch out 60 Hz noise, the signal was sampled at 120 Hz and 120 consecutive samples were averaged (Porat, B., A Course in Digital Signaling Processing: 164-167. John Wiley & Sons, New York, 1997). The gain of the instrumentation amplifier was set to give a sensitivity of approximately 0.2 V/kPa, and the system was calibrated in a sealed vessel with an adjustable u-tube water manometer.

### Example 1

### Measuring Glucose with Closed-Reactor Sensor

A. Theory

The sensitivity of a manometric sensor using the depletion of a dissolved gas may be derived in a manner similar to that of a sensor using the liberation of a dissolved gas (Jenkins et al., 1999). A mass balance on the dissolved gas in the system yields:

$$[ \frac{{P}_{g ( i )} {V}_{g}}{RT} + {β}_{( i )} {V}_{1} ] - [ \frac{{P}_{g ( f )} {V}_{g}}{RT} + {β}_{( f )} {V}_{1} ] = α {V}_{1} \qquad ( 1 )$$

where P_{g} is the partial pressure of the dissolved gas, β is the concentration of dissolved gas in the sample, α is the concentration of dissolved gas required to react with the analyte in the sample, V₁ is the volume of the sample, V_{g} is the volume of gas adjacent to the sample, R is the universal gas constant, and T is absolute temperature. Subscripts containing (i) represent the given quantity before the reaction with the dissolved gas, and those containing (f) represent the given quantity after the reaction with the dissolved gas. Assuming that the dissolved gas is in equilibrium in the system before and after the chemical reaction is allowed to occur, one may relate β to P_{g} using Henry's Law:

β=P_{g}K_{H} (2)

where K_{H} is an empirical constant for the dissolved gas in the given sample. Substituting equation 2 into equation 1 and rearranging, the vacuum (ΔP) developed in the system is:

$$Δ P = {P}_{g ( i )} - {P}_{g ( f )} = \frac{α {V}_{1} RT}{{V}_{g} + {K}_{H} {V}_{1} RT} \qquad ( 3 )$$

Inspection of this equation shows that the sensitivity of the device is adjustable over a broad range by changing the volume of gas in the system relative to the sample volume. By making the volume of gas small relative to the sample volume, the theoretical detection limit can be made small. To illustrate this, consider the enzymatic oxidation of glucose where 1 molecule of O₂ oxidizes each molecule of glucose. Assuming a gas volume negligible compared to the sample volume, a K_{H} value of 1.25×10⁻⁸ M/Pa for O₂ at 25° C., and a pressure sensor precise to 10 Pa (the measured precision of the sensors used in this research), the theoretical detection limit would be 0.125 μM, or about 22.5 ng/ml.

B. Materials and Methods

The fluid handling and instrumentation used for this example were similar to those previously used for the urea sensor. The hardware for fluid handling in the sensor (FIG. 2) consisted of a bank of pinch valves (161P011, Neptune Research Inc., West Caldwell, N.J.) on a common manifold from which reagents could be pumped through a positive displacement diaphragm pump with a nominal stroke volume of 50 μl (120SP 12 50-4, Bio-Chem Valve Inc., Boonton, N.J.) into a reaction cell made from Delrin. By energizing either a bleed or a waste pinch valve on the reaction cell (225P011-21, Neptune Research Inc.), the cell could be filled or flushed. The tubing used was a silicone based tubing made for the pinch valves (Neptune research Inc., TBGM107, 0.8 mm ID upstream of pump, and TBGM101, 1.5 mm ID downstream of pump). When the waste and bleed valves were closed, depletion of oxygen from the gas into solution could be measured as a change in vacuum in the reaction cell using a 10 kPa piezoresistive pressure sensor (MPX2010DP, Motorola, Phoenix, Ariz.).

To measure glucose, 5 strokes of the sample were loaded into the reaction cell with 15 strokes of an enzyme solution containing glucose oxidase (EC #1.1.3.4). The reaction cell was then sealed and shaken with a small DC motor for 30 seconds during which the change in vacuum was recorded. This shaking time was taken to be sufficient to observe most of the rapid changes in vacuum in the system. The stroke volume of the pump was measured as 37 μl, and the volume of the reaction cell including dead space in the adjacent tubing was measured to be about 1.2 ml. The enzyme solution was prepared with 2 mg/ml of glucose oxidase isolated from Aspergillus niger (Product # G7141, 245.9 units/mg, Sigma Aldrich Chemical Corp., St. Louis, MO.) and 1 mg/ml of peroxidase from horseradish (EC #1.11.2.7; Product # P8125, 116 purpurogallin units/mg, Sigma Aldrich Chemical Corp.) dissolved in a citrate/ascorbate buffer (50 mM citric acid and 34 mM ascorbic acid, pH 5.4). The glucose oxidase was used to oxidize β-D-glucose to δ-D-gluconolactone and peroxide, and the peroxidase was used to peroxidate acorbic acid, thus preventing the spontaneous decomposition of peroxide to oxygen and water. To prepare standards for analysis, 50 mM citrate buffer of pH 5.4 was used to dissolve D-glucose. To allow the mutarotation of glucose to come to equilibrium, the standards were left out overnight at room temperature. The wash solution used was distilled water, which was pumped through the reaction cell and waste and bleed lines after each analysis in a wash cycle. The pressure and temperature in the reaction cell were recorded: the reaction cell was ported to the negative port of the pressure sensor in order to record vacuum.

C. Results and Discussions

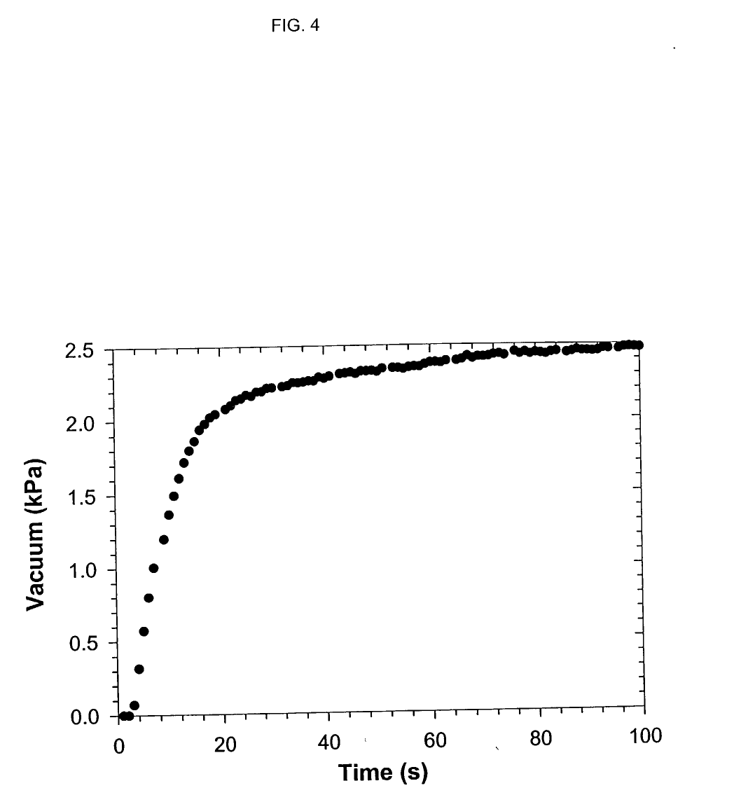

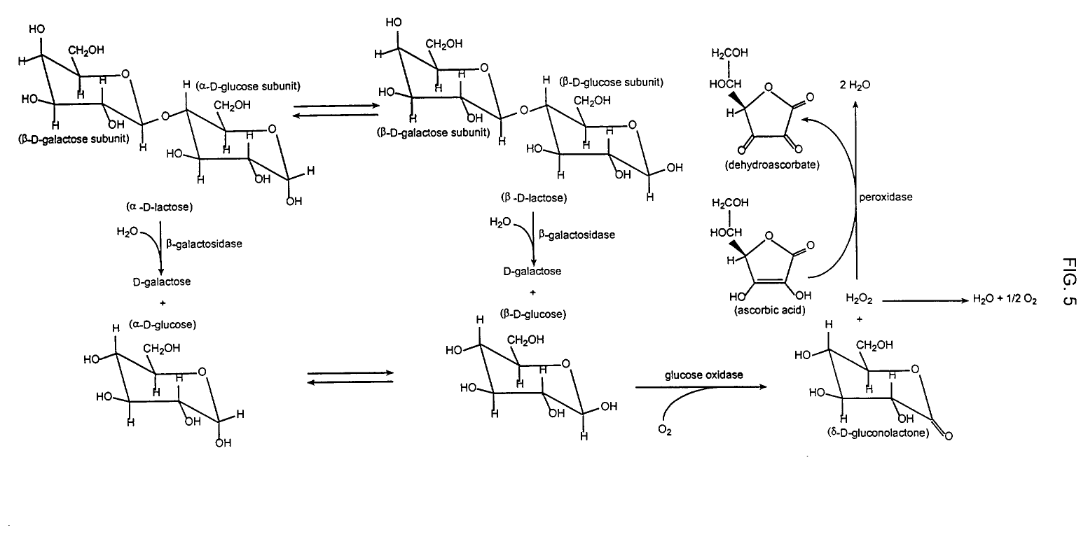

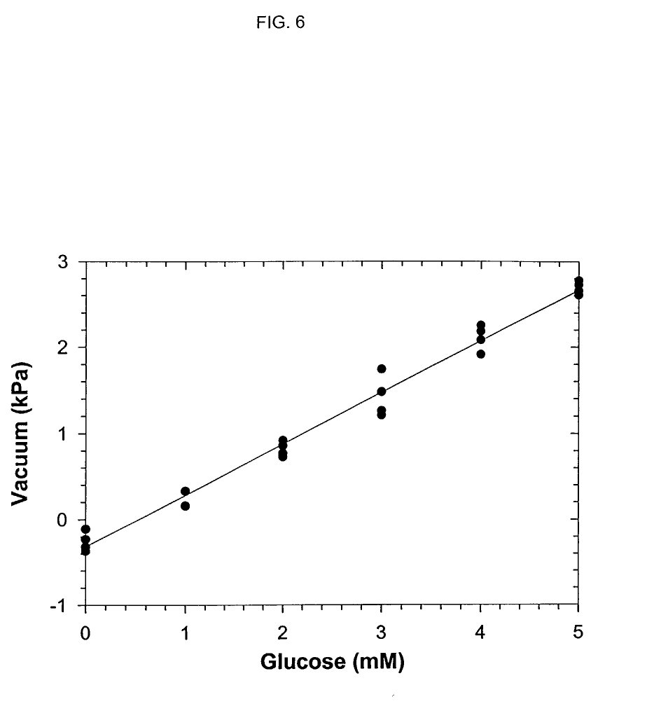

The manometric sensor was reasonably accurate for measuring glucose (FIG. 6). Variations in pressure change recorded for standards of the same concentration (FIG. 6) were larger than errors in pressure observed with a similar sensor for urea. These deviations from previous research were partly due to the spontaneous mutarotation of glucose (FIG. 5). Because glucose oxidase is only active on the β-D-glucose isomer (see, Keilin and Hartree, Biochem. J 50:341-348, 1952), any glucose in the (α-D-glucose conformation could not be detected. Inspection of the vacuum profile observed over a sample (FIG. 4) reveals that the pool of available β-D-glucose was exhausted shortly after introduction of glucose oxidase. When this occurred, the oxidation of glucose was limited by the rate of mutarotation of the α-D-glucose isomer to the β-D-glucose isomer. In the absence of a biological catalyst, the rate constant for the mutarotation of glucose at 20° C. has been measured as 0.015 min⁻¹ in pure water (see, Keilin and Hartree, supra; Livingstone et al., J. Solution Chem. 6: 203-216,1977) and has been observed to range from 0.006 to 0.186 min⁻¹ in various aqueous solutions (see, Keilan and Hartree, supra; Keilan and Hartree, Biochem. J 50:341-348, 1952; Livingstone et al., supra; Pigman and Isbell, Advan. Carbohyd. Chem. 23:11-57, 1968). The transition to this mutarotation limited reaction rate caused the abrupt change in the rate of change of vacuum over the sample (FIG. 4). The ratio of observed to predicted sensitivity (62.8%) was similar to the amount of D-glucose measured to be in the β configuration at equilibrium (62.6%), suggesting that little of the α-D-glucose was converted to β-D-glucose during the 30-second reaction period in the sensor. Because the reaction was not allowed to proceed to completion, errors were introduced by variations in the rates of mutarotation, mass transfer of gas into solution, and enzymatic oxidation of glucose.

### Example 2

### Measuring Lactose with Closed-Reactor Sensor

A. Materials and Methods

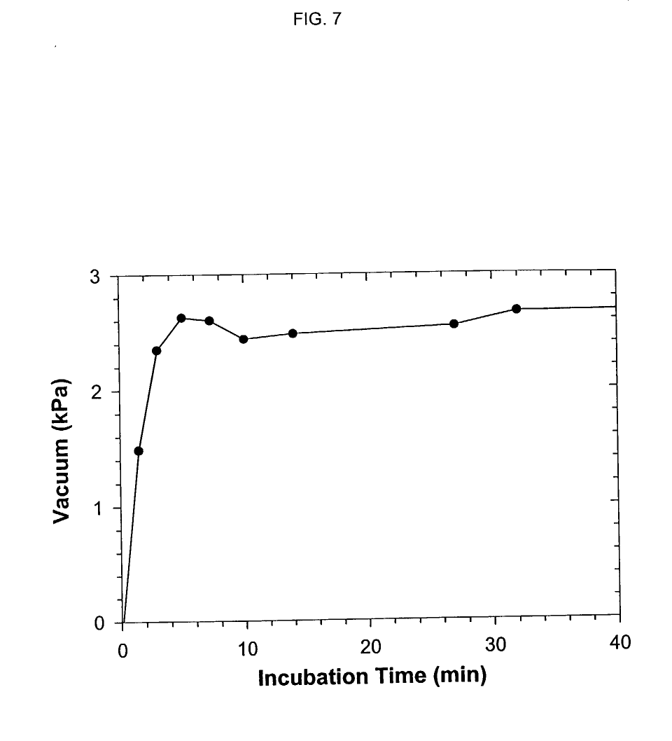

Lactose was determined by first hydrolyzing the lactose to D-glucose and D-galactose with the enzyme β-galactosidase (EC #3.2.1.23), and subsequently measuring the glucose as described above (FIG. 7). The β-galactosidase used was an industrial preparation isolated from Aspergillus oryzae (Product # G5160, 8.7 units/mg, Sigma Aldrich Chemical Corp.) that was homogenized with dextrin. Because dextrin was shown to interfere analytically with the determination of glucose, steps were taken to separate the dextrin from the enzyme prior to its application in the lactose assay.

To separate the dextrin from β-galactosidase, 1.5 g of the commercial preparation were dissolved into 20 ml of EDTA buffer (10 mM EDTA, pH 6.9) along with 150 mg of α-amylase from porcine pancreas (EC #3.2.1.1; Product # A3176, 12.3 units/mg α-amylase activity, 3.8 units/mg β-amylase activity, Sigma Aldrich Chemical Corp.) to hydrolyze the dextrin into maltose. The resulting enzyme stock was sealed in dialysis tubing (Spectra/Por regenerated cellulose membrane, 12,000 to 14,000 molecular weight cutoff, 16 mm diameter, Spectrum Laboratories, Rancho Dominguez, Calif.), and dialyzed three times for 2 hours in 11 of the EDTA buffer at room temperature to remove maltose. The solution was then saturated with ammonium sulfate to precipitate the enzyme, which was separated by centrifugation (5 minutes at 14,000 g). The resulting protein pellet was redissolved in 5 ml of a 50 mM citrate buffer of pH 4.5.

Lactose standards were prepared by dissolving D-lactose into the pH 4.5 citrate buffer. To allow the mutarotation of lactose to come to equilibrium, these standards were left out overnight at room temperature. The standards were incubated at room temperature with an aliquot of 1 volume of the purified β-galactosidase stock per 10 volumes of standard for at least 30 minutes. These were then assayed for glucose as described in the section above. Because milk solids tended to separate spontaneously during the incubation with the enzyme, milk samples were centrifuged to remove these components prior to the assay. These clarified samples were diluted 50 times into the pH 4.5 citrate buffer, then assayed for lactose as described in Example 1. To estimate the lactose concentration of the whole milk, the lactose estimated from the calibration equation was multiplied by the dilution factor (50) and corrected for the removal of fat and protein. For comparison, milk lactose was also measured using a Fourier transform infrared (FTIR) instrument (DairyLab 1, Foss Electric, Hillerod, Denmark). The same instrument was used to estimate the milk fat and protein in order to do the lactose correction for the manometric sensor.

B. Results and Discussion

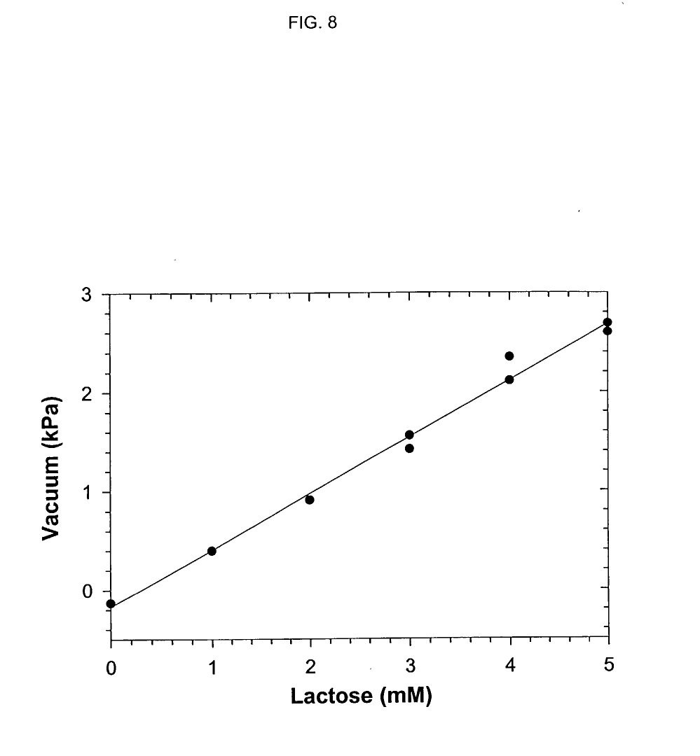

The 30-minute incubation time of lactose standards with β-galactosidase was shown to be adequate to hydrolyze all of the lactose to glucose and galactose, and in fact the incubation period could have been made as short as 5 minutes without any loss of sensitivity (FIG. 7). The sensitivity (0.572 kPa/mM) and precision (±0.19 mM) for the assay of lactose standards at 25° C. (FIG. 8) were similar to the respective values in glucose (0.597 kPa/mM and 0.23 mM in FIG. 6). The slightly smaller sensitivity for lactose may have been partially attributable to the slight dilution of the lactose standards with β-galactosidase solution prior to assay. Because the vaporization of water leads to pressurization in the sensor, the differences in the intercepts of the calibration equations may have been due to differences in atmospheric humidity on the days that the standards were assayed. The slopes of the calibration equations, however, were reproducible, so that the equations could be estimated by assuming a slope and assaying a single standard.

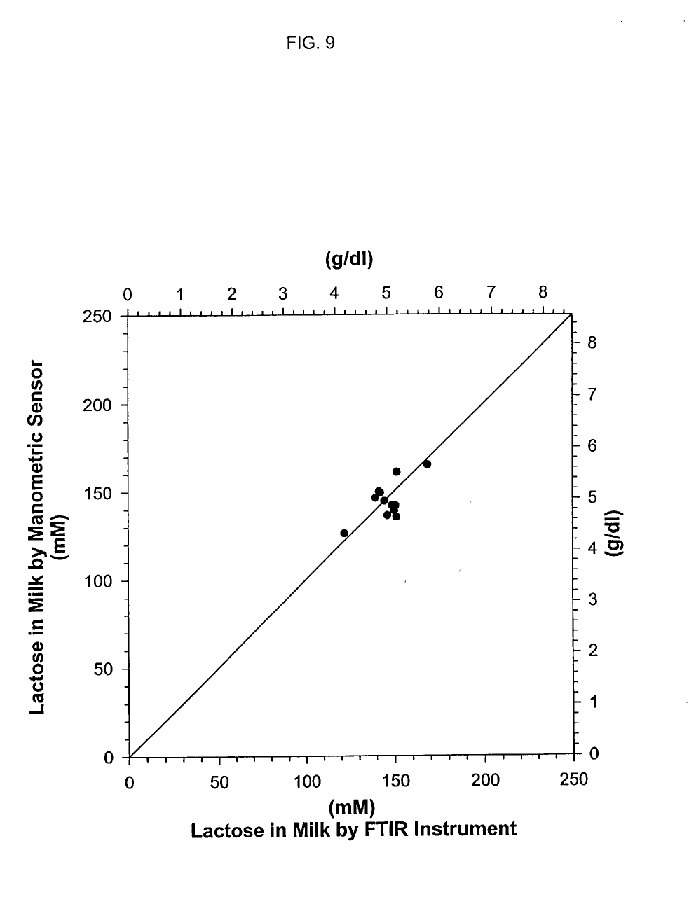

The estimate of lactose in milk was shown to be reasonably accurate compared to the reference FTIR instrument (FIG. 9). The correlation did not appear very strong (R²=0.45) because the milk samples collected had similar lactose concentrations. However, the root mean squared deviation of the two methods (8.1 mM) was only 5.8% of the average milk lactose value, showing that the two methods agreed well. Some of the observed error may have been attributable to the FTIR instrument. According to the distributor, the accuracy of the Dairy Lab 2 FTIR instrument is ±1.2 mM for lactose in milk (calculated from Foss North America, Dairy Lab 2 (Specifications). Eden Prairie, Minn., USA, 2001).

### Example 3

### Estimation of Humidity with Improved Sensor

A. Theory

a) Bubbling point within a Porous Membrane

The key component of the immersible manometric sensor was a porous membrane within which gas could be maintained at a pressure independent of the bulk pressure of the liquid outside of it. If the gaseous cavity was pressurized relative to the liquid, then the surface energy of the liquid (γ) on a hydrophilic membrane could contain a up to a critical pressure differential, or bubbling point, P* within the cavity. This pressure would occur when the surface of the liquid was deformed into a hemisphere bounded by the edge of the pore, having a diameter D equivalent to that of the pore. Performing a force balance on this hemispherical bubble,

$$\begin{matrix}\frac{π {D}^{2} {P}^{*}}{4} = π D γ so that & ( 4 ) \\ {P}^{*} = \frac{4 γ}{D} & ( 5 )\end{matrix}$$

Assuming a surface tension of 71.2 mN/m for pure water at 30° C., then a wetted membrane with 1 μm pores could, in theory, contain 284 kPa before allowing gas to bubble through. As long as the contact angle of the liquid on the membrane was less than 90° (the membrane was hydrophilic), the theoretical bubbling point through the pore would depend solely on the surface energy and the pore diameter, and not the degree to which the membrane was hydrophilic.

b) Vapor pressure deficit as a means to determine Atmospheric Humidity

If a porous membrane was fixed over a gaseous cavity and then immersed into distilled water, the gaseous cavity would pressurize until it was saturated with water vapor. The degree to which the cavity pressurized would be equivalent to the difference between the saturation vapor pressure at the temperature of the water and the vapor pressure of water in the atmosphere. These could be determined from the wet and dry bulb temperatures (T_{Wb} and T_{db}) and atmospheric pressure (Pₐₜₘ):

$${P}_{sat ( T )} = {ⅇ}^{( \frac{16.78 T - 116 \; 9}{T + 2373} )} \qquad ( 6 )$$

Pᵥ=P_{sat(Twb)}−APₐₜₘ(T_{db}−T_{wb}) (7)

where P_{sat(T)} is the saturation pressure at any temperature T, P_{V} is the water vapor pressure in air, and

A=00066(1.0+000115T_{wb}) (8)

with all pressures in kPa and temperatures in °C. Assuming the water is at the same temperature as the atmosphere, the vapor pressure deficit in the sensor should be equivalent to the difference between P_{sat(Tdb)} and Pᵥ.

c) Change in dissolved gas concentration

The pressure in the membrane covered cavity described above would also change with changes in dissolved gas concentrations. The change in pressure, ΔP, could be predicted for an increase in a soluble gas or for a decrease in soluble gas by performing a mass balance on the dissolved gas in the sample and cavity and assuming no mass transfer between the system and the atmosphere:

$$Δ P = \frac{α {RT}_{abs}}{( {V}_{g} / {V}_{1} ) + {K}_{H} {RT}_{abs}} \qquad ( 9 )$$

where R is the universal gas constant, T_{sabs} is absolute temperature, K_{H} is Henry's solubility constant describing the concentration of a dissolved gas at equilibrium with a given partial pressure of the gas, V_{g} is the volume of the gaseous cavity, V₁ is the volume of the sample, and α is the concentration of gas liberated in the sample (a negative quantity if gas is consumed).

If the ratio of cavity volume to sample volume were lowered far below the product K_{H} R T, then the sensitivity to changes in the gas concentration would reach a maximum value dependent only on the solubility constant:

$$\frac{Δ P}{α} = {K}_{H}^{- 1} . \qquad ( 10 )$$

On the other hand, if the ratio of cavity volume to sample volume were increased, the sensitivity would be diminished because a relatively greater mass exchange of gas would be required to affect the same change in partial pressure in the cavity. For gases with a low solubility, such as oxygen, the product K_{H} R T is low and it can be difficult to achieve the maximum sensitivity. By constraining the gas to a constant volume cavity within a porous membrane, the desired ratio of cavity volume to sample volume could be achieved for the maximum sensitivity. Furthermore, because the cavity volume is constant, the precision of the sensor could be improved over sensor types requiring dispensation of precise volumes into a reactor.

B. Materials and Methods

To estimate humidity, a sample of distilled water was allowed to come into thermal and chemical equilibrium with the atmosphere by agitation in a glass flask. The pressure monitor was then immersed in the water and the pressure change after 3 minutes was recorded. The observed pressure change was compared to the vapor pressure deficit predicted based on theoretic calculation. The wet and dry bulb temperatures were recorded using a commercial battery-powered psychrometer with Fahrenheit thermometers (Psychro-Dyne Model #3312-20, Cole-Parmer Instrument Company, Vernon Hills, Ill., USA), and atmospheric pressure was read on a mercury barometer. The vapor pressure estimated by subtracting the observed pressure change in the immersible monitor from the saturation vapor pressure at the dry bulb temperature was also used to estimate relative humidity, or the ratio of vapor pressure to the saturation vapor pressure at the dry bulb temperature. This was compared to the relative humidity determined purely from the psychrometric data.

No corrections were made for changes in vapor pressure due to the shape of the water surface interfacing with the gas. Condensation of water is favored onto a concave surface as compared to a planar surface because it causes a decrease in surface area, and therefore surface energy, on the concave surface (see, Adams, N.K., The Physics and Chemistry of Surfaces (2^{nd} Ed.) pp. 13-14. Clarendon Press, Oxford, UK, 1938). Therefore, the observed vapor pressure over a convex water surface is lower than that for a planar surface. Considering an energy balance for the reversible and isothermal distillation of water into a pool with a planar surface from a spherical droplet (adapted from experimentally verified relationships in Adam, supra, p. 14):

$${P}_{v}^{′} = {P}_{v} {e}^{- \frac{γ M}{ρ DRT}} \qquad ( 11 )$$

where P_{V} is the vapor pressure above the convex surface of diameter D, and O and M are the density and molecular weight of water. Assuming a temperature of 30° C. and a convex surface of diameter 1 μm, Pᵥ would differ only 0.05% from Pᵥ. Consequently, the effects of surface shape were not considered, even though they could become important with smaller pore sizes.

C. Results and Discussion

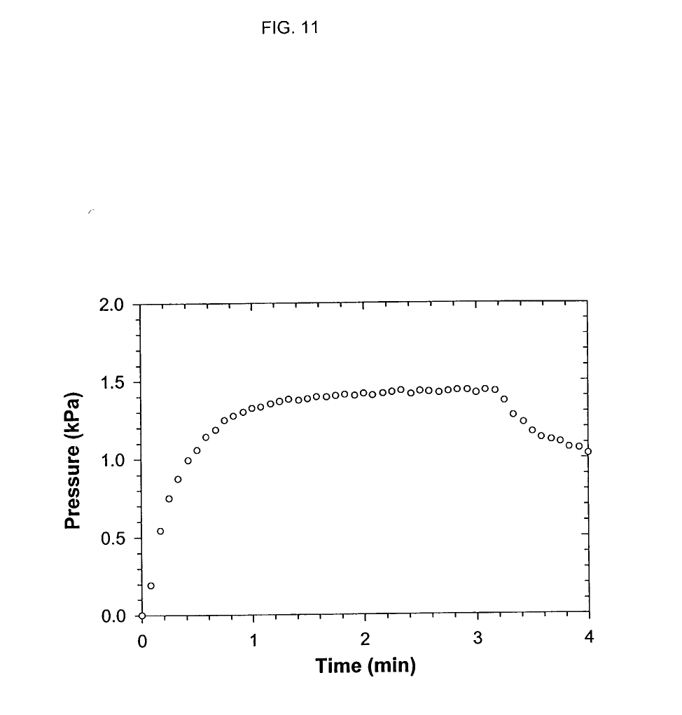

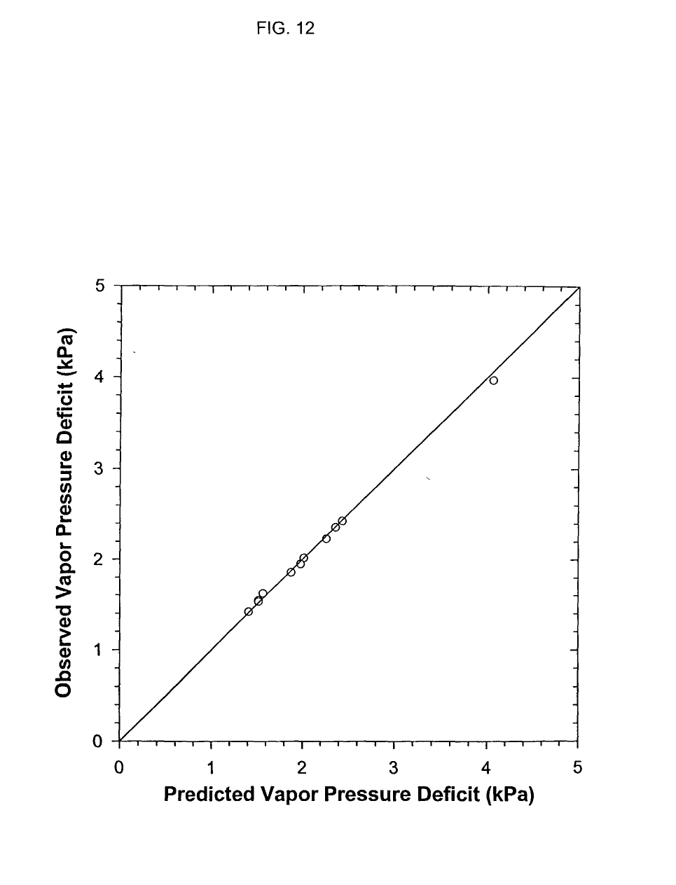

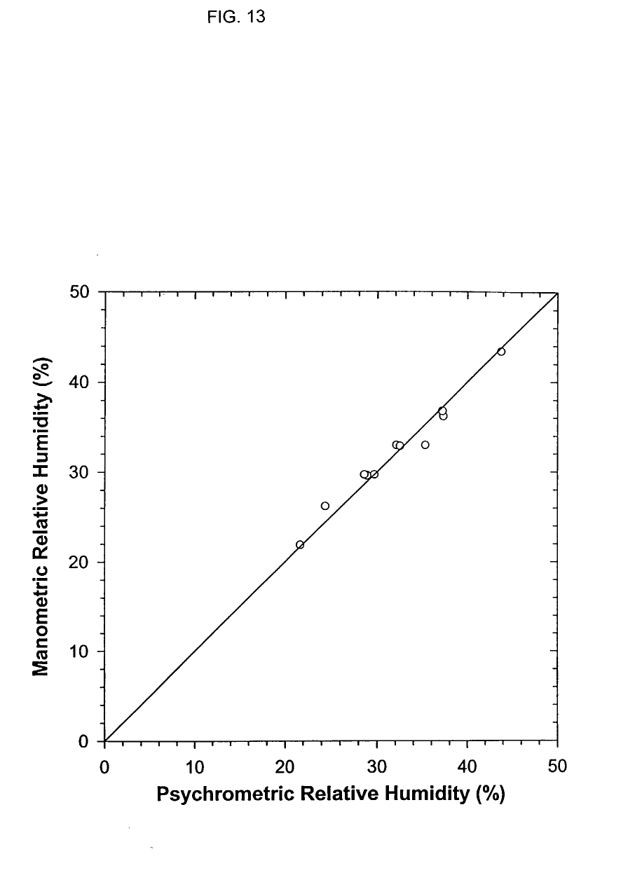

A typical record of pressure recorded after immersion of the pressure monitor in water (FIG. 11) showed that the saturation vapor pressure was reached well within the 3-minute time period. The observed pressure change in the monitor was well correlated to the vapor pressure deficit predicted from the psychrometric data (FIG. 12; R²=0.9993; root mean squared difference =0.04 kPa). Predictions of relative humidity based on the immersible pressure monitor data compared to predictions based on psychrometric data (FIG. 13; R²=0.9737; root mean squared difference =1.1%) were not quite as close due to the small range of relative humidity observed and the compounding of errors from pressure and temperature observations. Dry bulb temperatures for these tests ranged from 20.6 to 34.2° C.

Using the improved immersible pressure monitor, relative humidity could be estimated to within about 1% of predictions based psychrometric observations. By injecting samples of air into a cavity adjacent to a wetted membrane, a modification of the technology may prove useful for determining humidity in extremely small samples.

### Example 4

### Continuous Monitoring of Dissolved CO₂ with Improved Sensor

A. Materials and Methods

To monitor changes in dissolved CO₂ of a solution, the pressure monitor was immersed in 250 ml of 50 mM HCl solution in a glass flask open to the atmosphere. The solution was continuously stirred over a stir plate with a magnetic stir bar. After allowing the cavity to come to equilibrium with water vapor and other dissolved gases in solution, 1 ml injections of 250 mM bicarbonate solution were delivered into the solution at timed intervals. This caused step changes of approximately 1 mM in the CO₂ concentration of the solution. The pressure was recorded at 5-second intervals throughout the entire process to observe the changes in cavity pressure in response to changes in CO₂ concentration.

B. Results and Discussions

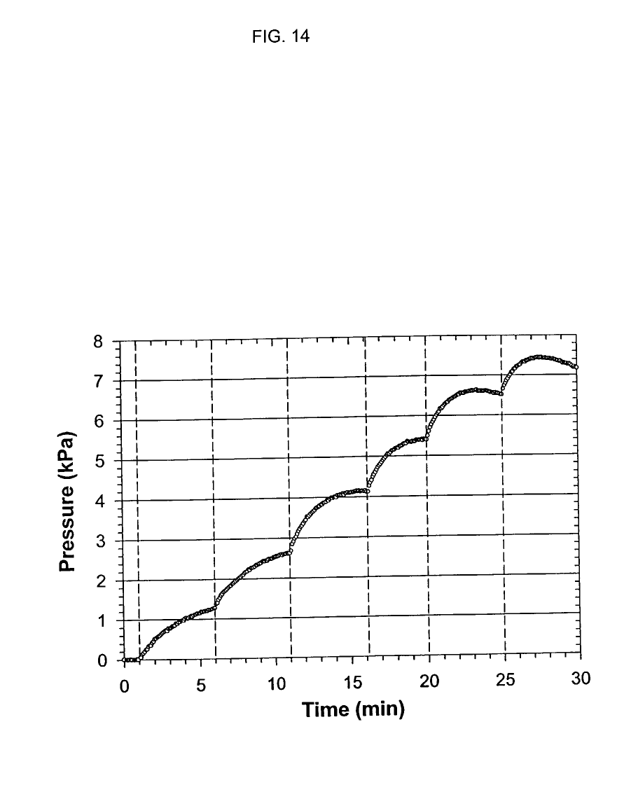

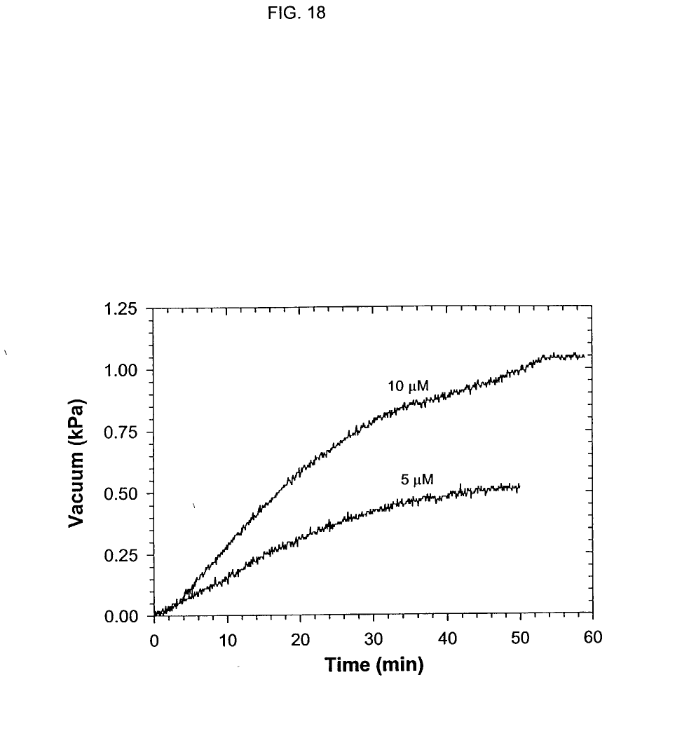

Clear changes in pressure were observed in response to step changes in the dissolved CO₂ concentration in solution (FIG. 14). Based on observation of the data, the approximate sensitivity of the pressure monitor to a change in CO₂ concentration was about 1.4 kPa/mM, which was close to the value of 1.33 kPa/mM observed at 24° C. by Jenkins et al. (J. Dairy Sci. 82:1999-2004, 1999) for the sensitivity of a manometric assay of CO₂ generated from the enzymatic hydrolysis of urea when the gas volume was 0.44 times the sample volume. Projections based on theoretic calculation and the CO₂ solubility observed in this previous work suggest that the maximum sensitivity for the CO₂ sensor would be about 1.74 kPa/mM at 24° C. The discrepancy between the apparent sensitivity observed here and the projected value indicate that a substantial amount of gas exchange occurred with the atmosphere since the system was not sealed. Further evidence of this is apparent in FIG. 18. Typical profiles of vacuum over time observed during the assay of glucose standards, suggesting that the 60 min pressure development time may not have been sufficient to reach equilibrium. as pressure was actually observed to be lost from the cavity when the CO₂ content was already high and several minutes elapsed since the last step increase in CO₂. It was for this reason that more care was taken to seal the vessel from the atmosphere when determining urea or glucose.

This technology is useful for monitoring a variety of processes or fluid streams since it was demonstrated that the pressure monitor could continuously monitor dissolved gas concentrations. Changes in sensitivity with temperature could be corrected in software. Differences in solubility of the target dissolved gas in different samples could be controlled by slight dilution with a concentrated electrolyte. For example, differences in CO₂ solubility were not significantly different between milk samples, buffer and distilled water when these samples were diluted by 17% with a 1 M citric acid solution.

### Example 5

### Measuring Urea with Improved Sensor

A. Materials and Methods

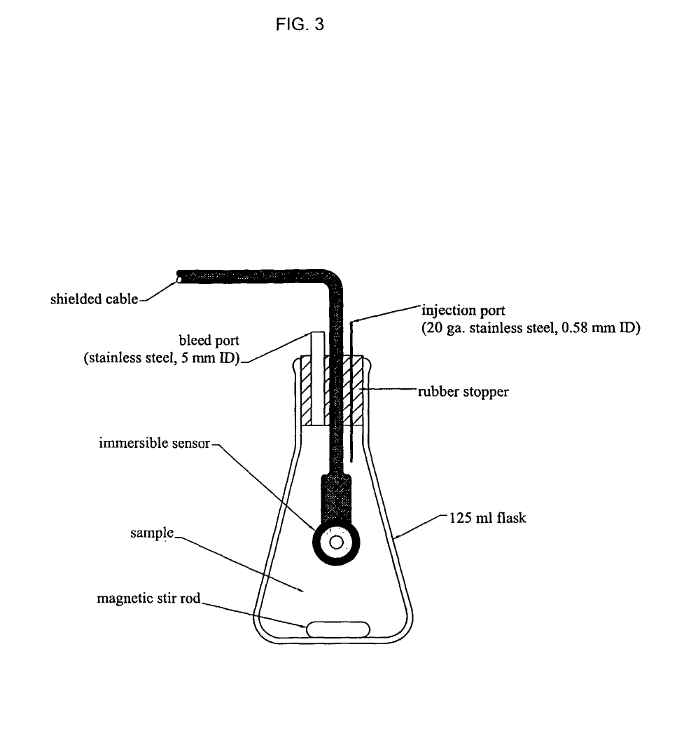

Urea standards were prepared in a 10 mM EDTA buffer of pH 7.2. Lyophilized urease (U4002, nominally 62.1 units/mg, Sigma Aldrich Chemical Corp., St. Louis, Mo., USA) was added to the standards to give 4 μg urease per ml. The standards were incubated with urease for 1 hour. To assay one hydrolyzed standard, a 125 ml flask was filled with the standard and sealed with a rubber stopper housing the immersible pressure monitor cable, an injection port, and a bleed port (FIG. 3). The stopper was placed slowly and carefully over the solution to prevent any sudden increases in pressure in the solution which might cause a loss of integrity in the pressure monitor cavity and to prevent the entraining of bubbles into the system. When the monitor came into equilibrium with the solution, 1 ml of a 2.5 M citric acid solution was injected through the injection port using a 1 ml pipette with a silicone tubing attachment. The injection and bleed ports were then covered with a paraffin film (Parafilm, American National Can, Neenah, Wis., USA) to prevent further gas exchange with the atmosphere. The injection port was made of much smaller diameter than the bleed port so that most of the pressure drop due to the injection occurred across the injection port, thus preventing any sudden pressurization of the sample which might cause the monitor to lose integrity. The injection of citric acid caused a lowering of the solution pH below 4, effectively converting the ionized forms of carbonate generated from the hydrolysis of urea to dissolved CO₂. The pressure was monitored every 5 seconds with continuous magnetic stirring, and the pressure change was recorded after 15 minutes or after the pressure failed to change more than 0.04 kPa over 2 contiguous minutes, whichever occurred sooner.

B. Results and Discussions

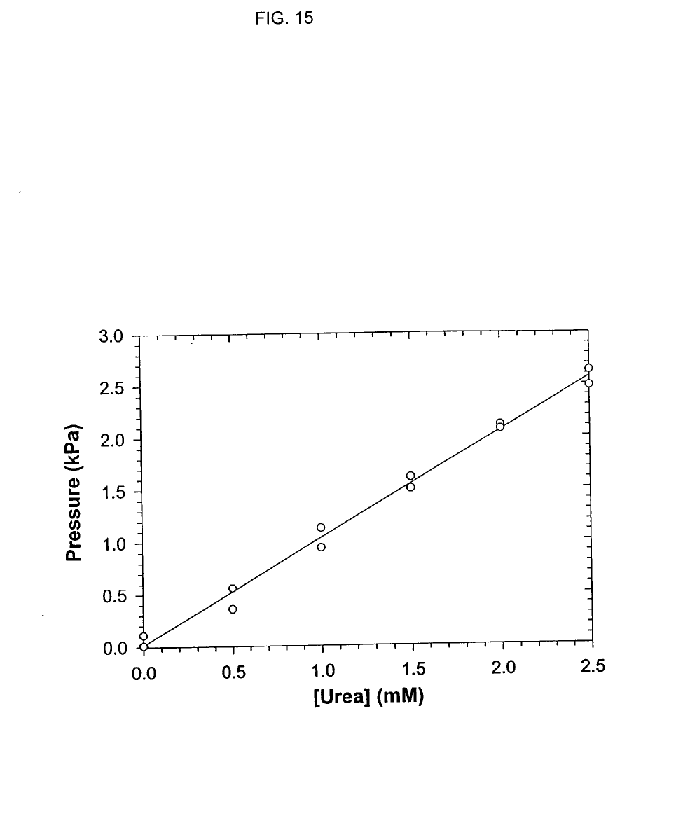

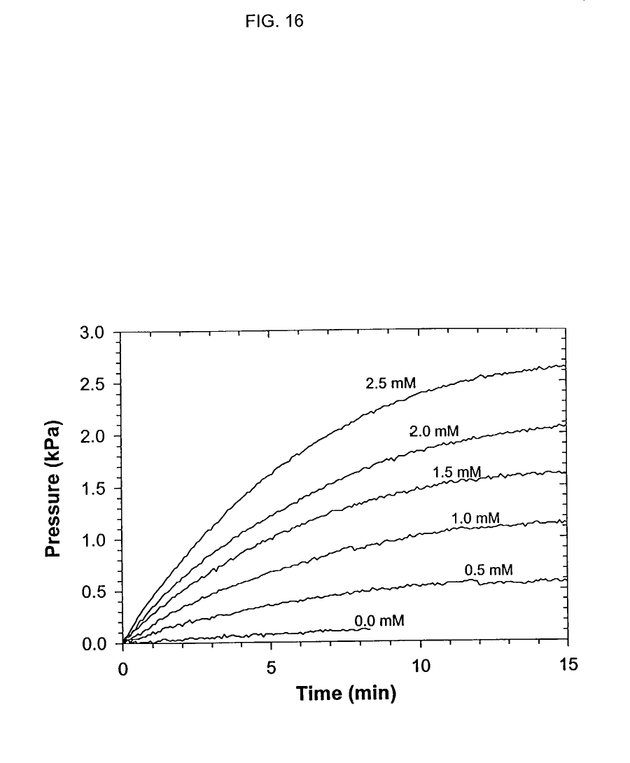

The standard curve for observed pressure change against urea concentration at 28° C. (FIG. 15; R²=0.9917; standard error =0.086 mM) showed that urea could be estimated reasonably accurately, but the sensitivity (1.022 kPa/mM) was less than that predicted based on the information above even at a lower temperature (1.74 kPa/mM at 24° C.). Inspection of typical profiles of pressure during the urea assay (FIG. 16) revealed that the mass transfer into the membrane in the urea assay was too slow to observe the full sensitivity during the 15-minute development time. The sensitivity and accuracy may have been improved by allowing more time for pressure to develop in the system, and some differences in sensitivity may have occurred from previous projections due to differences in CO₂ solubility in the buffers used.

### Example 6

### Measuring Glucose with Improved Sensor

A. Materials and Methods

Glucose was assayed with the same apparatus used in Example 5. D-Glucose standards were prepared in a pH 5.1 citrate-ascorbate buffer (25 mM citrate, 25 mM ascorbate, 150 mM NaCI) and left covered at room temperature overnight to allow the mutarotation between α-D-glucose and β-D-glucose to come to equilibrium. The 125 ml flask was then filled with the standard and sealed with the rubber stopper housing the pressure monitor, observing the same precautions as for urea. After allowing the monitor to come to equilibrium with the solution, 1 ml of an enzyme stock containing 5 mg/ml of glucose oxidase (EC# 1.1.3.4, Catalog # G7141, nominally 245.9 units/mg, Sigma Aldrich Chemical Corp.) and 1 mg/ml of peroxidase (EC# 1.11.1.7, Catalog # P 8125, nominally 116 units/mg, Sigma Aldrich Chemical Corp.) in citrate-ascorbate buffer was injected into the flask. The injection and bleed ports were then covered with paraffin film, and the solution was continuously stirred with a magnetic stir bar for 60 minutes or until the pressure failed to change by more than 0.04 kPa for 2 minutes. The pressure change, which was negative and thus recorded as vacuum, occurred due to the consumption of oxygen by the enzymatic oxidation of glucose. Peroxidase was added to the enzyme solution to catalyze the redox reaction between ascorbic acid and the peroxide generated during the oxidation of glucose (Jenkins and Delwiche, Adaption of a Manometric Biosensor to Measure Glucose and Lactose, submitted to Biosensors & Bioelectronics, 2001), thereby prevented the decomposition of peroxide to oxygen and water. This was done as a precaution because enzyme preparations frequently contain biological impurities that accelerate the decomposition of peroxide because it is toxic to many cells.

B. Results and Discussions

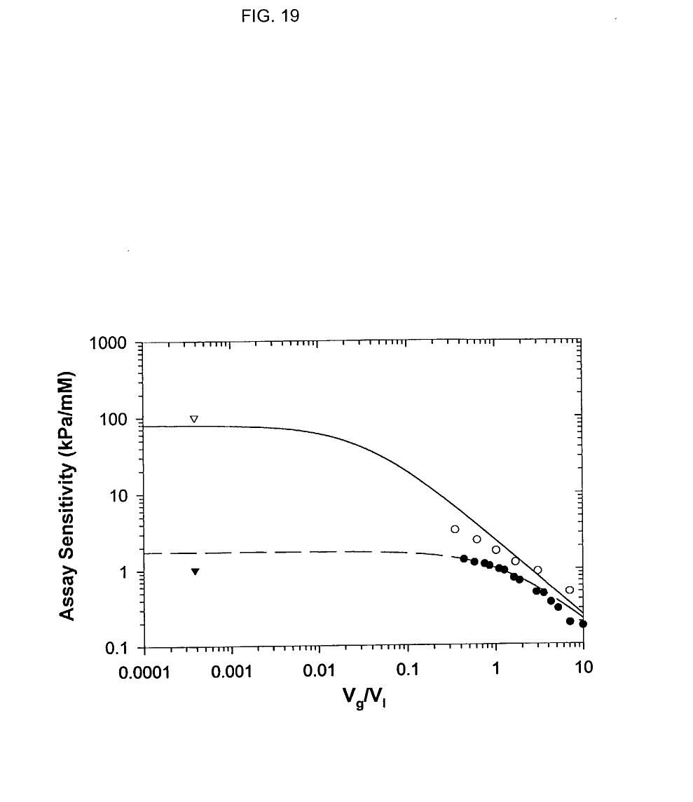

The sensitivity of the sensor for glucose was 101.2 kPa/mM (FIG. 18), which was much higher than that for urea. This was due to the fact that oxygen is much less soluble than CO₂ . Because of this increase in sensitivity the standard error was observed to be only 0.76 μM, or about 137 ng/ml. A typical profile of vacuum observed during an assay of glucose (FIG. 19) showed that 60 minutes was not long enough to observe the full change in pressure, and that even greater sensitivity may be observed with improved mass transfer or longer vacuum development times.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A method for assaying a liquid for the presence of a component said method, comprising:
(a) contacting said liquid with a sensor comprising a gaseous chamber in contact with a pressure transducer, wherein said gaseous chamber is separated from said liquid by a permeable membrane; and
(b) detecting a change in pressure in said gaseous chamber with said pressure transducer, wherein said change in pressure is due to the presence of said component in said liquid.

2. The method according to claim 1, wherein said liquid and said sensor are contained in a sealed vessel.

3. The method according to claim 1, wherein said detecting is continuous.

4. The method according to claim 1, wherein said liquid is a component of a process or a stream.

5. The method according to claim 1, further comprising:
(c) degrading said component to generate a gas, thereby causing a positive pressure change.

6. The method according to claim 5, wherein said degradation is enzymatic degradation.

7. The method according to claim 6, wherein said component is urea, said enzyme is urease and said gas is carbon dioxide.

8. The method according to claim 1, further comprising:
(c) degrading said component to deplete a gas in said liquid, thereby causing a negative pressure change.

9. The method according to claim 8, wherein said component is glucose, said enzyme is glucose oxidase and said gas is oxygen.

10. The method according to claim 8, wherein said component is lactose, said enzyme is glucose oxidase and said gas is oxygen, said method further comprising:
(d) hydrolyzing lactose into glucose and galactose.

11. The method according to claim 14, wherein said hydrolyzing is by β-galactosidase.

12. The method according to claim 1, wherein said fluid is a member selected from blood, milk and urine.

13. A method of detecting a change in concentration of a component in a liquid, said method comprising:
(a) contacting said fluid with a sensor comprising a gaseous chamber in contact with a pressure transducer, wherein said gaseous chamber is separated from said fluid by a permeable membrane; and
(b) detecting a change in pressure in said gaseous chamber with said pressure transducer, wherein said change in pressure is proportional to said change in said concentration of said component in said fluid.

14. The method according to claim 13, wherein said detecting is continuous.

15. The method according to claim 13, wherein said liquid is a component of a process or a stream.

16. A method of detecting humidity in a system comprising a fluid, said method comprising:
(a) contacting said fluid with a sensor comprising a gaseous chamber in contact with a pressure transducer, wherein said gaseous chamber is separated from said fluid by a permeable membrane; and
(b) detecting a change in pressure in said gaseous chamber with said pressure transducer, wherein said change in pressure is proportional to said humidity.

17. A sensor for detecting the presence or absence of a dissolved gaseous component in a liquid, said sensor comprising:
a housing defining a chamber with an upper section comprising an opening communicating with the atmosphere, said opening covered by a porous membrane having an inside surface; and
a lower section, comprising a piezoresistive pressure transducer covered with a transducer coating material having an upper surface,
wherein said inside surface of said membrane and said upper surface of said coating material define a gaseous cavity.

18. The sensor according to claim 17, further comprising an encapsulant coating said housing.

19. The sensor according to claim 17, further comprising a connection between said piezoresistive transducer and instrumentation external to said sensor for transmitting data from said transducer to said instrumentation.
